(19) United States
(12) Patent Application Publication
(10) Pub. No.: US 2022/0354982 A1
(43) Pub. Date: Nov. 10, 2022

(54) AUTOMATED METHOD FOR IMPLEMENTING UV-C LIGHT FOR AIR PURIFICATION IN HOTEL ROOMS

(71) Applicant: Jeffrey Benjamin Johns, Las Vegas, NV (US)

(72) Inventor: Jeffrey Benjamin Johns, Las Vegas, NV (US)

(21) Appl. No.: 17/307,331

(22) Filed: May 4, 2021

(51) Int. Cl. A61L 9/20; F24F 8/22; F24F 3/16; F24F 11/30

(52) U.S. Cl. CPC A61L 9/20; F24F 8/22; F24F 3/16; F24F 11/30; A61L 2209/111; A61L 2209/16; F24F 2120/10

(57) ABSTRACT

An automated method for implementing UV-C light for air purification in hotel rooms is disclosed. By implementing a method for identifying the hotel room rental status and occupancy status, the UV-C light can be automated to a specific cycle time that will not cause issues with guest complaints or potential safety issues.

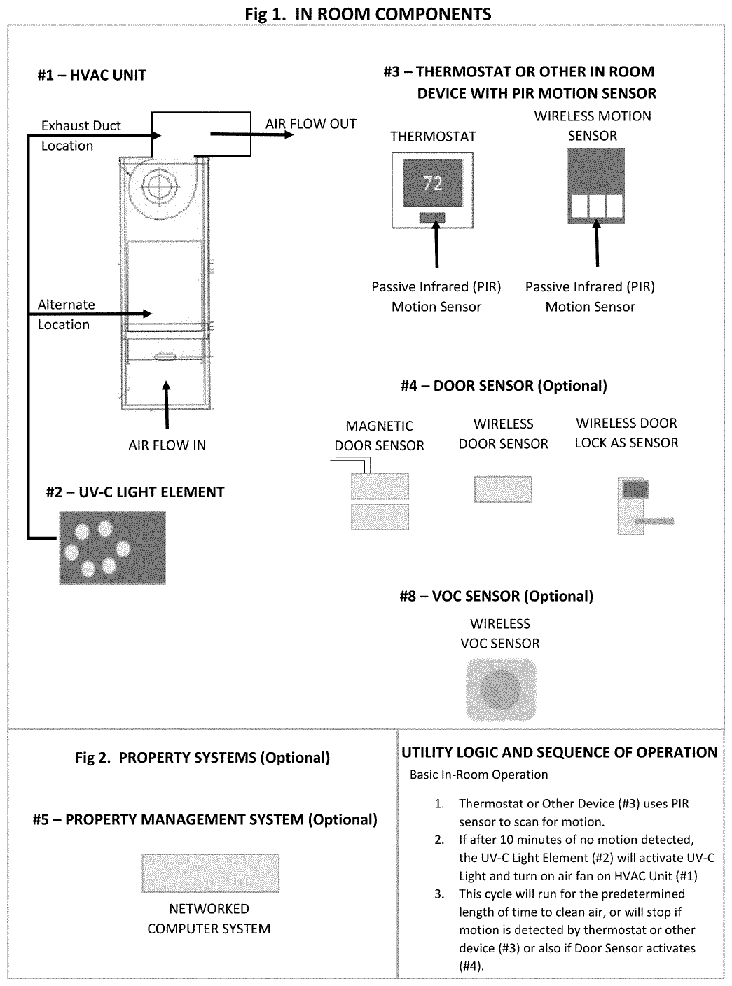

### BACKGROUND

Embodiments of the invention described in this specification relate generally to air purification systems, and more particularly, to an automated method for implementing ultra-violet (UV-C) light for air purification in hotel rooms.

Indoor air quality is a major health concern especially in locations for high turnover of occupancy, such as a hotel room that may have hundreds of different individuals living and sleeping in one room over the course of a year.

The current solutions are focused on the residential homes, and are constantly powered. A residential system is generally in an attic area and any UV-C light leakage would not be seen. Hotel rooms have the HVAC units inside each room, and it is not practical to run the UV-C light constantly. At night blue UV-C light leakage could come from the HVAC unit causing guest complaints.

Therefore, what is needed is a way to automatically determine when a hotel room is not occupied by a person and to sanitize the hotel room with a UV-C light when there is no person in the hotel room.

### BRIEF DESCRIPTION

A novel automated method for implementing UV-C light for air purification in hotel rooms is disclosed. By implementing the method for identifying the hotel room rental status and occupancy status, a UV-C light in a hotel room can be automated to a specific cycle time that will not cause issues with guest complaints or potential safety issues.

### BRIEF DESCRIPTION OF THE DRAWINGS

Having thus described the invention in general terms, reference is now made to the accompanying drawings, which are not necessarily drawn to scale, and which show different views of different example embodiments.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

Some embodiments of the invention include a novel automated method for implementing UV-C light for air purification in hotel rooms. By implementing a method for identifying the hotel room rental status and occupancy status, the UV-C light can be automated to a specific cycle time that will not cause issues with guest complaints or potential safety issues.

As stated above, indoor air quality is a major health concern especially in locations for high turnover of occupancy, such as a hotel room that may have hundreds of different individuals living and sleeping in one room over the course of a year. Embodiments of the automated method for implementing UV-C light for air purification in hotel rooms described in this specification solve such problems by retrofitting of a standard HVAC air unit in a hotel room with a UV-C light element, combined with an automated process of identifying that a room is unoccupied, such that the system can activate the UV-C light and HVAC fan for a duration of time to sanitize the indoor air for the room.

Embodiments of the automated method for implementing UV-C light for air purification in hotel rooms described in this specification differ from and improve upon currently existing options. In particular, typical hotel room HVAC systems do not include UV-C lighting for sanitary air. However, there are some conventional UV-C fixtures that are available and targeted for home use. The improvement provided by the automated method for implementing UV-C light for air purification in hotel rooms is using key methods of hotel room data from networked property systems and occupancy detection to activate the UV-C light when the room is determined to be unoccupied. This addresses human safety from UV-C lighting issues and timing for defined sanitary cycles.

In addition, some embodiments of the automated method for implementing UV-C light for air purification in hotel rooms improve upon the conventional options because the method of identifying cycles for UV-C light treatment of indoor air does not exist in the hotel room category. This means the conventional UV-C light solutions are not practical for hotel rooms. Not only is it dangerous to run UV-C light-based air purification when a person is present in a hotel room, but it can also be less effective. Thus, by implementing a method for identifying the hotel room rental status and occupancy status, the UV-C light can be automated to a specific cycle time that will not cause issues with guest complaints or potential safety issues.

The automated method for implementing UV-C light for air purification in hotel rooms of the present disclosure may be comprised of the following elements. This list of possible constituent elements is intended to be exemplary only and it is not intended that this list be used to limit the automated method for implementing UV-C light for air purification in hotel rooms of the present application to just these elements. Persons having ordinary skill in the art relevant to the present disclosure may understand there to be equivalent elements that may be substituted within the present disclosure without changing the essential function or operation of the automated method for implementing UV-C light for air purification in hotel rooms.

1. Hotel room HVAC unit (heating, ventilation, and air conditioning)

2. UV-C light element to sanitize indoor air

3. Thermostat or other in-room device with a passive infrared motion sensor for occupancy detection

4. Door sensor (optional) to improve accuracy of occupancy detection

5. Property Management System (PMS) (optional) to send system status of Rented or Un-rented

6. Room Rental Status (optional)—The current rental status of a hotel room

7. Room Occupancy Status—The current occupancy status of a hotel room

8. Volatile Organic Compound (VOC) sensor—Optional sensor to measure the room VOC and activate system.

The various elements of the automated method for implementing UV-C light for air purification in hotel rooms of the present disclosure as presented in the figures and in this Specification may be related in the following exemplary fashion. It is not intended to limit the scope or nature of the relationships between the various elements and the following examples are presented as illustrative examples only.

#1 is the hotel room air conditioning unit that will run based on temperature and other controls.

This unit has fan motor typically with High, Medium and Low speeds.

#2 is the UV-C Light element that is mounted inside the HVAC unit (#1). This light element can be powered by multiple voltages based on available power.

#3 is the thermostat or other in-room device with a passive infrared (PIR) motion sensor that detects occupancy to control the HVAC Unit (#1) and can activate the UV-C light (#2) based on a sequence of operations. The PIR sensor determines if motion has been seen in the room.

#4 is an optional door sensor that can be a magnetic sensor or other device that informs the thermostat (#3) that a potential change in occupancy is occurring. If the UV-C light (#2) is running, and a door sensor send a signal to thermostat (#3) of the door opening, the UV-C light (#2) can be shut off for safety.

#5 is an optional Property Management System (PMS) is a networked computer system used by hotels to manage the rental status and other details of guest rooms. The front desk staff would check-out a room when the guest departs, and the PMS would notify the thermostat (#3) of the rental status (#6) and that the room is Unrented. If the thermostat or other in-room occupancy detection device (#3) also determines the room to be unoccupied (#7) the system could start the UV-C light (#2) and HVAC (#1) fan for air sanitization.

#6 is the rental status of a specific room as identified by the networked Property Management System (#5). Rooms can be set to various statuses, including checked-in, checked-out, checked-out dirty, checked-out clean, out of order. The sequence of operations of the UV-C light (#2) and HVAC (#1) functions can be based on room status.

#7 is the occupancy status of a room. If a room is occupied, the system would not activate the UV-C light (#2) to avoid potential guest complaints and safety concern of UV-C light. A room can have a status of Occupied or Unoccupied. The thermostat with PIR sensor or other in-room device (#3) is the primary device for determining occupancy by motion detection.

#8 is an optional sensor designed to measure and report the volatile organic compounds (VOC) in the air. This can be used to trigger the system of UV-C light (#2) and HVAC (#1) when VOCs are too high and room Occupancy Status (#7) is determined to be unoccupied. Without this optional sensor, the system can be operated based on programmed cycle times and sequence of operations.

The automated method for implementing UV-C light for air purification in hotel rooms of the present disclosure generally works by implementing UV-C light sanitation in hotel rooms using a specific method that is both safe and effective. While conventional systems are designed for residential uses as a continual function, the automated method for implementing UV-C light for air purification in hotel rooms of the present disclosure integrates and functions with existing hotel systems to provide an application of current systems to solve for indoor air quality issues. Also, by combining Rental Status, Occupancy Status, a thermostat or other in-room device with PIR sensor and UV-C light element in the HVAC unit, the automated method for implementing UV-C light for air purification in hotel rooms provides a way to safely implement UV-C light treatment in a hotel room. Based on one or both status of Rental Status (#6) and Occupancy Status (#7) the UV-C light (#2) cycle will run the HVAC unit (#1). Checking logic states will affect the operation of the system.

In some embodiments, it is possible to have a system without a Property Management System (#5), in which case the automated method for implementing UV-C light for air purification in hotel rooms will only rely on the thermostat or other in-room device with PIR sensor (#3). As such, a room determined to be unoccupied by the PIR sensor for a defined period of time will activate the UV-C light (#2) and HVAC (#1) fan. This will run for a predetermined period of time to sanitize the indoor air. During this operation of the UV-C light (#2) and HVAC (#1) fan, if occupancy is detected by the PIR sensor (#3), the cycle will stop and turn off UV-C light (#2). However, if there is a PMS system (#5), the cycle can be set as a function of room rental status (#6). This could start the UV-C light (#2) and HVAC (#1) fan after a room is changed to a room rental status (#6) Unrented Clean, and Room Occupancy Status (#7) of unoccupied. In some embodiments, activation and logic for the timing of the UV-C light (#2) and HVAC (#1) fan cycle is controlled by the thermostat with PIR sensor (#3). The thermostat (#3) triggers the UV-C light (#2) by activating a relay in the HVAC unit (#1) and the thermostat (#3) activates a specific fan speed in the HVAC unit (#1). In some embodiments, an optional Door Sensor (#4) will send commands either by contact relay or by wireless signal to the thermostat (#3) that a door has opened or closed. The logic of a door opening, closing, and the thermostat or other in-room device with PIR sensor (#3) detecting motion will set the room occupancy status (#7) to occupied. In some embodiments, if acurrently occupied room is provided a door sensor (#4) signal that the door opened and closed, then the thermostat or other in-room device with PIR sensor (#3) will scan for occupancy for a predetermined amount of time. After scanning for this predetermined amount of time, if no motion is detected, then the room occupancy status (#7) will be set to unoccupied.

To make the automated method for implementing UV-C light for air purification in hotel rooms of the present disclosure, a standard HVAC fan coil unit in a hotel room can be retrofitted with a powered UV-C light element designed and rated for the automated method for implementing UV-C light for air purification in hotel rooms. The power source for the UV-C light element would be wired to the control of the fan coil using a compatible voltage (24 VAC is most common). This can be wired to a fan speed terminal that is disabled, or other available terminal that provides the voltage. The thermostat or other in-room device with PIR sensor would be wired to the terminals for these controls. The logic sequence for the control of the UV-C light would be programmed into the thermostat to control the details of rental status, occupancy and other elements. There are specific hotel-designed thermostats that include flexible logic programming that can control the system. If networked and connected to the PMS system, the thermostat will have all the details necessary to control and activate the UV light and HVAC fan unit.

In some embodiments, the basic components of the system require a HVAC unit (#1) with an airflow chamber, the UV-C light (#2) element and a thermostat or other in-room device with PIR sensor (#3). The other items are optional, yet make the system more accurate and precise as an operational practice in performing the automated method for implementing UV-C light for air purification in hotel rooms. The door sensor (#4) provides greater accuracy of occupancy by working in conjunction with the thermostat and PIR sensor (#3). It also provides an immediate trigger upon opening of the door to change room occupancy (#7) from unoccupied to occupied. The Property Management System (#5) integration into the system allows for network visibility to room status (#6) and also for staff to see UV-C light (#2) status while tracking and reporting on UV-C light run cycles by room. The UV-C light (#2) and HVAC (#1) fan cycles are made more efficient with greater detail on the room status, which can further define logic and sequence of operation. The volatile organic compound (VOC) sensor (#8) makes the system better by establishing measurements and feedback on the indoor air quality. Rather than just using time-based logic that is defined in advance, the VOC sensor (#8) can trigger the UV-C light (#2) and HVAC (#1) fan when measurements exceed acceptable levels and run the cycle until the VOC sensor (#8) reports back mitigation of the unacceptable levels. The VOC sensor (#8) also enables a way to benchmark and report the indoor air quality of each room. It is possible to change characteristics of components or logic methods, yet the key elements of the HVAC unit (#1), using a light source for sanitary treatment of indoor air such as by the UV-C light (#2), and a motion sensor for Occupancy Detection (#7) are the core elements. Yet, in some embodiments, a system could be developed that only uses a PIR motion sensor that is outside the thermostat (#3) or is independent to trigger a controller that resides with the UV-C light (#2). These would be alternatives designed to approximate the same automated method for implementing UV-C light for air purification in hotel rooms.

To use the automated method for implementing UV-C light for air purification in hotel rooms of the present disclosure, an operator of a hotel would either construct new or retrofit an HVAC unit with the provided UV-C light device and wire this according to the design specific for their HVAC system. The software logic in the thermostat would be updated to implement the sequence of operations specific to their room and network system design. Testing of the time necessary to “turn over” the air volume in the room, and VOC levels before and after can be measured to customize the UV-C light cycle timing and HVAC fan speeds. From this stage, the system is automated and runs based on the logic of the automated method for implementing UV-C light for air purification in hotel rooms. If reporting is desired either by room or aggregated by property, this can be accessed from a web-based report by the operator.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

I claim:

1. An automated method for implementing UV-C light for air purification in hotel rooms, said automated method comprising:
identifying a fan motor of a heating, ventilation, and air conditioning (HVAC) unit that is installed in each hotel room of a hotel;
mounting a UV-C light element inside the HVAC unit or air duct work and powering the UV-C light element based on available power from the HVAC unit;
configuring a thermostat or other in-room device with a passive infrared (PIR) sensor that controls the HVAC unit inside the hotel room to activate the UV-C light element when no motion is detected inside the hotel room for a predetermined amount of time;
configuring a magnetic door sensor available at any hotel room to send a signal to the thermostat when the door opens, wherein the signal to the thermostat triggers the UV-C light to shut off for safety; and
integrating a property management network system with the thermostat, the UV-C light, and the HVAC and configuring the property management network system to notify the thermostat of a rental status of the hotel room, wherein when the thermostat is notified of the rental status as unrented and the thermostat determines that the hotel room is not occupied as indicated by no detected motion, the thermostat triggers the UV-C light to start for air purification and the HVAC fan to start for air sanitization.
